(19) United States
(12) Patent Application Publication
(10) Pub. No.: US 2009/0117168 A1
(43) Pub. Date: May 7, 2009

(54) Device and method for attaracting diseased cells and foreign substances

(75) Inventor: James Keenan, Ottawa (CA)

Correspondence Address: KNOBBE MARTENS OLSON & BEAR LLP, 2040 MAIN STREET, FOURTEENTH FLOOR, IRVINE, CA, 92614, US

(73) Assignee: H.E.F., Andrezieux Boutheon (FR)

(21) Appl. No.: 10/579,165

(22) PCT Filed: Nov. 9, 2004
(86) PCT No.: PCT/CA04/01940

§ 371 Date: Aug. 15, 2008

(30) Foreign Application Priority Data
Nov. 12, 2003 (CA) 2448995

(51) Int. Cl. A61F 2/02; A61K 33/26; A61P 35/00; A61P 31/12

(52) U.S. Cl. 424/423; 424/648

(57) ABSTRACT

An implantable device for attracting diseased cells or foreign substances circulating within bodily fluid channels is disclosed. The device comprises a frame, an attachment means for maintaining the frame in a localized position in a body vessel, and at least one attractant on the frame, where the attractant is capable of attracting a diseased cell or a foreign substance. The device may also include a therapeutic agent The device may be used for diagnosis, disease containment or treatment. The device may also be used for attracting and localized treatment of diseased cells and foreign substances.

### FIELD OF THE INVENTION

The invention generally relates to the attraction of cells or viruses circulating through blood vessels, lymph channels, and spinal fluid channels for localized therapy, disease containment, or diagnosis.

### BACKGROUND OF THE INVENTION

Disease may spread through a body via fluid channels. Disease may come in many forms from foreign substances such as viruses, bacteria, parasites and the like, to diseased cells, such as cancerous cells, infected or damaged cells. Once in fluid channels, such as the vascular system, lymph system or spinal fluid channels, these diseases may be able to circulate in a body. For example, most cancer deaths are caused by metastasis rather than primary tumours. The delivery of circulating cancer cells to secondary sites is generally regulated by bodily fluid flow and the molecular interaction between the cancer cells and the new organ environment.

A body has natural means to fight disease. White blood cells, called leucocytes, circulate in the vascular and lymphatic systems, locating and attacking foreign substance and diseased cells, as part of a body's immune response. Occasionally, the response is insufficient to destroy the foreign substances, which may take hold elsewhere in the body and proliferate.

A variety of therapeutic agents may be used. For example, in the case of cancer, chemotherapy drugs and ionizing radiation may be used to weaken or destroy cancerous cells. Two common chemotherapy administration options are: system-wide doses delivered intravenously, which target tumours and circulating cancer cells, and localized treatment of solid tumours. These therapeutic agents typically have negative side effects, such as weakening a body's immune system and/or destroying healthy cells along with cancerous cells. Cancer patients undergoing system-wide chemotherapy treatment typically have their white blood cell count diminished.

Similarly, viruses are infectious agents that proliferate within cells. For most animal viruses, proliferation commences with attachment to a host cell, mediated by viral attachment proteins that recognizes and interact with proteins, called receptors, on a suitable host cell. Once viral components are inside the host cell, viral replication occurs. Certain viruses may circulate within the body until a suitable host cell having an appropriate receptor is found, whereupon the virus may proliferate.

Bacteria may also circulate within a body. Upon finding a suitable environment and under appropriate conditions, bacteria may reproduce rapidly and in some cases, produce damaging toxins and creating an infection.

Treatments with anti-viral and antibiotic agents are often system-wide and hence, non-specific, although some localized external applications are available. Biotherapeutics, especially small molecule proteins, may be used for more localized treatments but tend to have a limited half-life, or effective drug releasing time span, within the body.

Accordingly, there is a need for a means to facilitate the localization of diseased cells and foreign substances in vivo. Such means may be used for disease containment in vivo, for facilitating diagnosis, for facilitating localized application of therapeutic agents, or the like.

### SUMMARY OF THE INVENTION

The present invention is directed to an implantable device suitable for use in attracting diseased cells or foreign substances such as viruses, bacteria, parasites, other microbes, and the like (hereafter collectively “foreign substances”).

Accordingly, in an aspect of the present invention; there is provided a frame, attachment means for maintaining the frame in a localized position in a body vessel, and at least one attractant on the frame, wherein the attractant is capable of attracting a diseased cell or foreign substance within proximity to the frame. In some embodiments, the attractant is able to capture diseased cells or foreign substances, including by binding, electrical interaction, magnetic interaction, fusion, or the like.

A therapeutic agent may also be provided on the frame, the agent adapted to degrade cells or foreign substances attracted to the frame. The term “degrade” includes any degree of damage or destruction, or to render less operable, including by endocytosis or dissolution, or the like.

The device may be deployed in any body, mammalian or otherwise, having fluid channels.

The device may be employed to concentrate or capture circulating cells or foreign substances in proximity to a frame placed in a specified location for diagnosis, disease containment, or treatment. The device may be used on a wide variety of diseases, including viral, bacterial or parasitic infection or the like. It may be used to reduce or prevent metastasis by capturing cancer cells as they circulate in the body and preferably before they migrate to and invade distant organs.

In an embodiment, the frame comprises a radially stiff ring. This ring may also be magnetic.

In an embodiment, the attractant may be bound with endothelial cells, other cells, or agonist equivalents, thereby providing an environment for circulating cells or foreign substances to invade. The attractant may be physically separated from body vessels by the device or members or membranes provided on the device.

The device may be used to trigger a body's natural defenses, such as leukocytes (white blood cells), or may incorporate additional therapeutic agents to degrade diseased cells or foreign substances.

A highly localized dose of minute quantities of chemotherapy or brachytherapy may be applied to attracted circulating cancer cells, resulting in minimal side effects to healthy tissue.

An object of the disclosed invention is to provide a highly localized treatment of circulating cancer cells or other diseased cells or foreign substances. Localized micro-chemotherapy to treat metastasis with minimal side effects is not currently available.

The foregoing summarizes the principal features of the invention and some of its optional aspects. The invention may be further understood by the description of the preferred embodiments, in conjunction with the drawings, which follow.

### BRIEF DESCRIPTION OF THE DRAWINGS

The accompanying drawings illustrate presently preferred embodiments of the invention and, together with the description that follows, serve to explain the principles of the invention.

FIG. 1A depicts a perspective view of an embodiment of the invention deployed in a blood vessel.

FIG. 1B depicts a perspective view of another embodiment of the invention deployed in a blood vessel.

FIG. 2A depicts a preferred embodiment of the invention comprised of a magnetic ring.

FIGS. 2A, 2B, 2C, and 2D depict a sequence where the preferred embodiment is supplied with attractant substances and therapeutic agents in vivo, and captures and treats circulating cancer cells.

FIG. 3 depicts front view and side view of an embodiment of the invention comprised of an open ended ring with a member extending radially within the bodily fluid vessel.

FIGS. 4A and 4B depict an embodiment of the invention having a mechanical means to destroy captured cells, the means activated by an external energy source.

### DESCRIPTION OF THE PREFERRED EMBODIMENT

Reference will now be made in detail to various suitable embodiments of the invention as illustrated in the accompanying drawings. It will be understood that this description is exemplary and is to assist in understanding the invention and the principles of operation.

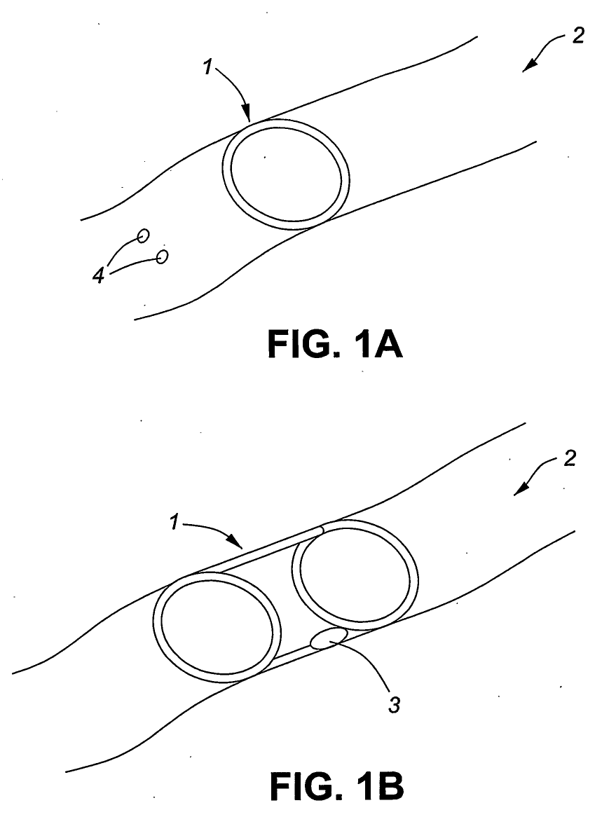

Referring to FIG. 1, an implantable device is provided for use in attracting diseased cells or foreign substances such as viruses, bacteria, fungi, parasites, microbes, or the like. The implantable device includes a frame (1) deployed within a patient's blood vessel (2). The frame (1) secures the device to the bodily fluid vessel at a selected location, and binds to it an attractant and a therapeutic agent. Circulating cancer cells (4) are migrating through the blood vessel.

As depicted in FIG. 1A, the frame (1) is in the form of a single ring that extends around the circumference of the bodily fluid vessel, such as an artery or vein, a lymphatic vessel and the like. The frame (1) is sized and shaped to frictionally contact the vessel to maintain a position and has bound to it an attractant and a therapeutic agent.

Alternatively, the frame (1) may be in the form of an open-ended ring, a plurality of rings joined with axial members such as depicted in FIG. 1B, a plurality of rings and axial members or struts in a knitted, zig-zag, open cell, closed cell, helical, or other pattern. As a further alternative, the frame may be in the form of a simple plug, sphere, pellet, or cylindrical shape.

Instead of frictional contact, the frame (1) may alternatively be embedded into a wall of a bodily fluid vessel with a portion of the device exposed to the bodily fluids or be provided outside a vessel with portions extending through a vessel wall for exposure to bodily fluids. Other means for maintaining the frame at a selected location in a vessel are contemplated.

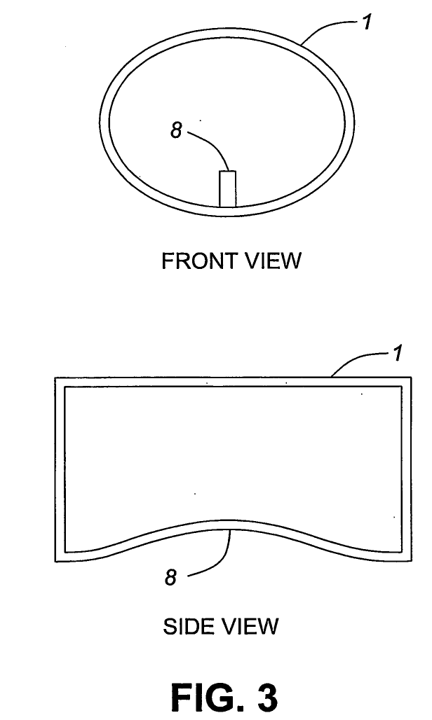

The frame (1) may be provided with members or membranes that extend down the bodily fluid vessel. The frame may consist of concentric rings or incorporate members that extend axially within the bodily fluid vessel, or extend radially inwards towards the centre of the vessel. FIG. 3 depicts another embodiment, comprised of a single ring (1), with a member (8) that extends radially inwards. Members may link opposing sides of the frame radially across the vessel.

Members extending radially inward helps to extend the therapeutic life of the device. A few weeks after implanting, the bodily fluid vessel lining, the endothelium, may grow over the perimeter surface of the frame, and so the portions of the device extending radially inward will remain exposed to the bodily fluid flow and circulating diseased cells or foreign substances.

Members extending axially down bodily fluid vessels helps to extend the therapeutic range of the device, and permit treatment with fewer devices implanted in a patient.

The frame may be flexible or rigid. Members or membranes attached to the frame may be flexible or rigid. The frame may be made of a wide variety of materials: for example, surgical-grade metal, plastic, ceramic, natural fibres, or a combination of materials, and may be formed in a variety of sizes. Struts may typically range from 0.1 to 4 mm in thickness, and the overall length of the device may typically range from 1 to 20 mm. The device dimensions may be lesser or greater than the typical range.

Alternatively, the frame may be made of a biodegradable material that over a period of months or years is absorbed and disappears. This would avoid long-term health and safety concerns associated with metallic or other non-biodegradable implants.

As a further alternative, the frame may be coated with bioactive treatments to improve device performance. Most materials can absorb a heparin type coating that reduces thrombus formation on blood-contacting devices. This form of coating can be used with other molecules to enhance cell attachment. Such a treatment may be applied by dip coating, flow-through, or spraying.

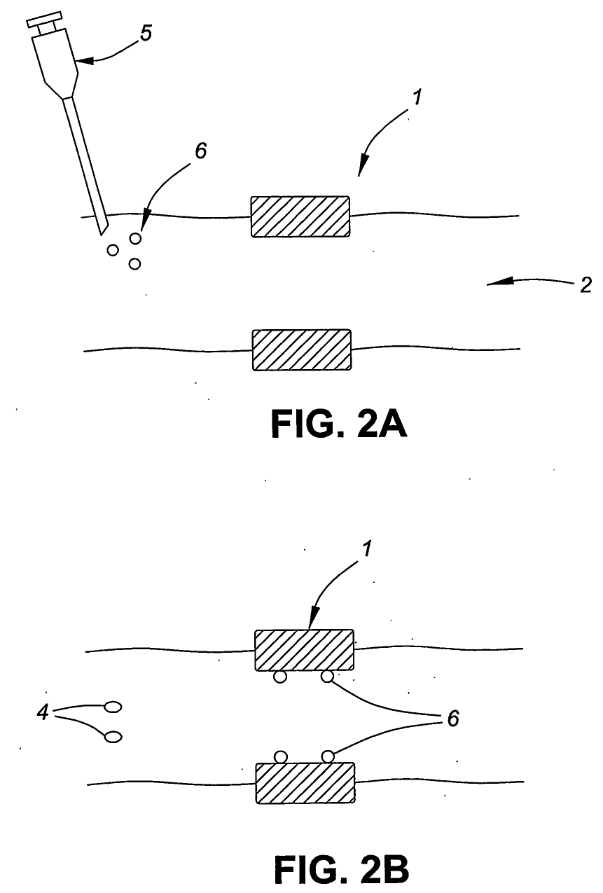

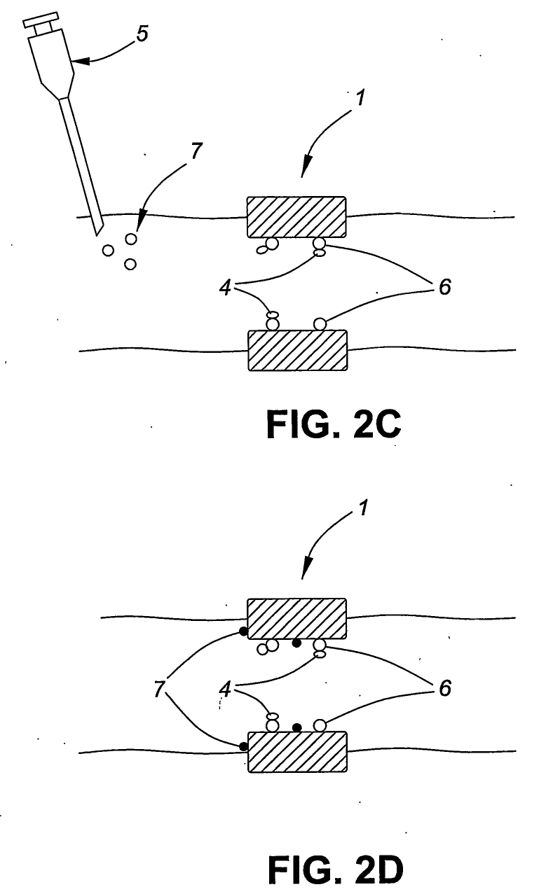

The frame or a portion of the frame may be magnetic. This includes the incorporation of at least one magnetic component, for example, a ferrous particle. FIG. 2A, depicts a presently preferred embodiment of the device comprised of a magnetized ring.

The frame may also incorporate physical features, surface treatments, markings, or coatings to enhance its visibility to imaging systems. Struts, or markings scribed into the frame surface, may be spaced to correspond to typical diagnostic ultrasound wavelengths in order to enhance the echogenicity of the device for ultrasound imaging. The device may elute MRI, X-ray, CT scan, ultrasound, or fluoroscopic contrast agents to enhance its visibility to those imaging systems.

Bound to the frame (1) is an attractant comprising one or more means to attract circulating cells or foreign substances through physical, chemical, biological, magnetic, electrical, or like means. Preferably, the attractant is able to capture or otherwise concentrate these cells or foreign substances proximate to the frame (1). Generally, the attractant is specific to a type of cell, a foreign substance, or a related group thereof.

Some substances have been found to attract cancer cells. For example, chemokines are a class of inflammatory induced, secreted proteins with the ability to attract and activate circulating leukocytes (white blood cells) and stem cells to home in on particular organs. Chemokines induce white blood cells to migrate towards them and to adhere to the endothelial cells lining blood vessels.

Studies have demonstrated that large quantities of chernokines released from certain organs also attract circulating cancer cells to invade.

Additionally, agonist drugs have been developed to mimic chemokines, to stimulate physiological activity at cell receptors normally stimulated by naturally occurring substances. Companies such as Chemokine Therapeutics design peptide analogs and peptidomimetics.

The attractant may be natural, i.e. produced by a body, such as chemokine proteins, neurotransmitters, viral host cell receptor proteins, or other natural receptors, and extracted for use. The attractant may be agonist drugs or chemical or biochemical formulations developed to simulate physiological activity at cell receptors, such as chemokine agonists which mimic chemokine proteins. Synthetic constructs may also be used. A combination of natural substances and/or agonist drugs and/or chemical or biochemical formulations or the like may be used.

The attractant may be coated, bound, combined or the like, with substances such as polymers in order to protect the attractant substances within the body and extend their time-release over prolonged periods.

Additionally, or alternatively, the attractant may be bound with other substances or components to provide a suitable environment for capturing circulating cells or encouraging foreign substances to invade. The invasive environment may, for example, consist of endothelial cells or their agonist equivalent, other cells, cellular by-products constructs other biotherapeutics or chemicals. Alternatively, these components may be directly deposited on, or otherwise contained on, the frame. The composition of the attractant and the invasive environment compounds may be formulated to capture specific types of diseased cells or foreign substances.

The frame (1) may elute the attractant and/or other drugs to aid treatment, improve biocompatibility, or enhance the external imaging of the device. Drug elution may be accomplished through coating the frame (1) with one or more attractant that leaches into the bodily fluid vessel or coating the frame (1) with attractant(s) imbedded in a thin polymer for time-release.

Another drug-elution method includes imbed drugs within a biodegradable frame or body. As the exterior of the frame is absorbed and disappears, drugs are eluted in a time-release manner.

Alternatively, the frame (1) may be formed with receptacles for storing quantities of attractant, which may be released in a time-released manner. Alternatively, there may be provided a containment that is activated on demand by an external energy source such as therapeutic ultrasound to release the attractant. FIG. 1B depicts an embodiment comprised of a frame (1) comprised of a plurality of rings, axial members, and a receptacle (3), deployed within a patient's blood vessel (2). The receptacle (3) contains and continually elutes a substance (not depicted) to attract and bind circulating cells or foreign substances.

Alternatively, the frame (1) may be embedded into a wall of a bodily fluid vessel with a portion of the device exposed to the bodily fluids, and the portions extending through a vessel wall may be comprised of or connected to receptacles for storing quantities of attractant.

Additionally, or alternatively, the attractant may be bound with ferrous particles, if magnetic means are used, to bind the attractant to the frame. The ferrous particles may be iron oxide as is used with commercially available magnetic cell separating equipment.

Additionally, the attractant may comprise an electronic component utilizing electrical properties for attraction.

Accordingly, it is to be understood that the concept of an attractant “on” or “bound to” a frame includes circumstances where the attractant is not physically “on” the frame and contemplates circumstance where the attractant is otherwise proximately connected to the frame, whether by a physical, magnetic, electrical or chemical connection.

An effective amount of attractant is to be used so as to manifest desired attraction properties. This amount would likely be in the order of micrograms, the desired quantity to be confirmed through routine trials.

The frame (1) may incorporate additional physical features, surface treatments, or coatings to enhance the capture of circulating cells, viruses or other foreign substances, or the like. The physical geometry of the device or a portion thereof, may be shaped and/or coated to mimic the physiology of organs, bone, or other tissues.

For example, the frame (1) may incorporate gold nanoparticles, which absorb laser light more thoroughly than other materials, and hence permit laser-assisted diagnostic imaging or laser ablation of captured cells or viruses.

Alternatively, the frame (1) may be formed of a plurality of components that are not physically connected but are proximately arranged in order to permit interaction.

Depending on the application, the circulating cells or foreign substances may localized, for example, concentrated in proximity to the frame or be captured onto the frame, for example, by binding, magnetic attraction, chemical interaction, electrical attraction or the like. The localized cells or foreign substances may be extracted for diagnostic purposes, retained in the location to facilitate disease containment, or for other purposes.

As depicted in FIG. 1, bound to the frame (1) is a therapeutic agent to degrade cells or foreign substances. As set out above, “degrade” includes any degree of damage, destruction, weakening or otherwise render less operable including by endocytosis or dissolution. The therapeutic agent may be an organic or inorganic compound, an ionizing radiation source, a laser source, a mechanical means to degrade, or a combination of different therapeutic agents. Alternatively, the therapeutic agent may comprise a therapeutic attractant, such as an antigen, a foreign chemical, lymphokines, or the like, to attract various immune system cells for a localized immune response therapeutically suitable for cells or foreign substances attracted to the frame. It is to be appreciated that, where lymphokines are desired to be used, an attractant suitable to attract the desired lymphokines to the device together with means to stimulate the lymphokine to attack the cells or foreign substances may be required. A plurality of therapeutic agents may also be used.

Comparable to the attractant, the therapeutic agent may be eluted from the frame (1). Elution may be accomplished through coating the frame (1) with one or more therapeutic agents that leaches into the fluid vessel or by coating the frame (1) with an agent(s) imbedded in a thin polymer for time-release. Alternatively, the therapeutic agent may be imbedded within a biodegradable frame. As the exterior of the frame is absorbed and disappears, drugs are eluted in a time-release manner.

Alternatively, the frame (1) may be formed with receptacles for storing quantities of therapeutic agent, which may be released into a vessel in a time-released manner. Alternatively, there may be provided a compartment or orifice that is activated on demand by an external energy source such as therapeutic ultrasound.

Alternatively, the frame (1) may be embedded into a wall of a bodily fluid vessel with a portion of the device exposed to the bodily fluids, and the portions extending through a vessel wall may be comprised of or connected to receptacles for storing quantities of therapeutic agents

The therapeutic agent(s) may also be bound with or to a ferrous or other magnetic particle, if magnetic means are used, to bind the therapeutic agent to the frame.

Where the therapeutic agent is an ionizing radiation source, alpha emitting isotopes may be used to degrade or destroy the captured diseased cells or viruses, notwithstanding its ordinarily limited ability to penetrate tissue (ie. a cell width). Close proximity may be achieved for a highly localized treatment with minimal damage to healthy tissue.

Alternatively, a laser or other high intensity source, acoustic source or electrical current or discharge, may be used to ablate or damage attracted cells or foreign substances.

Accordingly, it is to be understood that the concept of a therapeutic agent “on” or in association with a frame includes circumstances where the agent is not physically “on” the frame and contemplates circumstances where the therapeutic agent is otherwise proximately connected to the frame, whether by a physical, magnetic, electrical or chemical connection, or is applied onto or proximate to the frame, including by radiation thereon.

The treatments may be repeated or extended through replenishing the supply of the attractant substance(s) and/or the therapeutic agent(s), as the case may be. This will permit the attending physicians or veterinarians, or the like, to tailor the treatments to a patient's specific needs.

The frame may also form a physical barrier between the attractant substance and the patient's tissue. Therefore, the circulating diseased cells or viruses may invade the attractant substance, for a period of time before the therapeutic agent is applied, and not invade the patient

As mentioned, the device incorporated into vessels may continually elute substance(s), or may release substance(s) upon activation by an external energy source such as ultrasonic energy. Repeat treatments may be done through means such as intravenous or intra-arterial injection, with a biological, chemical, magnetic, electrical, or other physical means to bind the attractant substance(s), therapeutic Agent(s) or other drugs to the device.

An effective amount of therapeutic agent is employed, where the agent is a chemical, including drugs. In certain applications, for example, where circulating cancer cells have been attracted and are present in the local area in sufficient concentrations, a highly localized delivery of minute quantities of therapeutic agent(s) such as chemotherapy or brachytherapy may be sufficient. This will result in far less negative side effects to the patient than is common with most system-wide chemotherapy treatments.

The implantable device is generally deployed in bodily fluid vessels, for example, vascular vessels, lymphatic vessels, spinal vessels, or the like, where circulating diseased cells or viruses are known or suspected.

The device may be deployed within the bodily fluid vessel using a variety of means, including catheter, needle delivery, and manual placement in conjunction with surgery.

The device may be deployed as stents are deployed, using guide wires and angioplasty balloon catheters. The balloons come in a wide range of lengths and diameters, and are made from a variety of materials. The balloon portion of a catheter is inflated to expand the device radially outwardly into contact with the bodily fluid vessel wall, whereupon the device undergoes plastic deformation and remains in an expanded state at a fixed position within the bodily fluid vessel.

The device may be deployed as self-expanding stents are deployed, using catheters but no balloon. Thermal memory stents are deployed softened and compressed at a low temperature and return to their original shape, exerting a mild, continuous pressure on the bodily fluid vessel wall.

The device may be delivered via needle or probe, and may pierce the bodily fluid vessel to have a portion of the device exposed to the circulating diseased cells or viruses and a portion of the device used to anchor it in position.

Surgical placement of the device may be particularly advantageous to cancer treatment, as difficult to section tumours may be inadvertently set in circulation through the body through the surgical procedure itself.

A hybrid device, which accomplishes the aforementioned and in addition performs the function of a vascular stent, to help keep constricted blood vessels open, may also be used.

The implantable device may be used individually within a patient or a plurality may be used. A plurality of devices may or not be physically joined, some of which may contain one or more attractants, some of which may contain one or more therapeutic agents, and some of which may contain both one or more attractants to attract and capture circulating cells or viruses and therapeutic agent(s)

In the case of a patient diagnosed with metastasized cancer, the health care personnel may follow a treatment regime such as:
cycles of system-wide chemotherapy with external beam radiation or surgery or both
implant the device, or a plurality of the devices, within the body along confirmed or suspected routes of cancer cell circulation
use the device to capture and treat circulating cancer cells
replenish the attractant to attract and bind the circulating cancer cells and the therapeutic agent through intravenous injection as required for repeat treatments

The device can also be used to diagnose and stage other treatments by providing an indication of the quantity of captured cells using imaging systems and/or by retrieving a biopsy sample.

The disclosed device utilizes aspects of existing technology and techniques:
Design and deployment techniques for stent like devices implanted within bodily fluid vessels
Biotherapeutics such as chemokines and drug agonists such as chemokine mimics which attract and bind diseased cells
Procedures to protect and prolong the dosage time of biotherapeutics in vivo
Magnetic separation of targeted cells which bind to receptor cells bound to ferrous particles
Delivery of medicaments to specific blood vessels using intravenous or intra-arterial injection, needles, PortaCath™ type sheaths for repeat injection and other means
External energy sources activating medicaments at a depth within a patient

Embodiments and their operation may be illustrated by the following examples.

### EXAMPLE 1

In the embodiment of the device as shown in FIG. 1A, the frame is comprised of 316L stainless steel, laser cut, and polished. Its surface has been modified to enhance biocompatibility through a gas plasma treatment. The frame is coated with an attractant and a therapeutic agent

### EXAMPLE 2

FIGS. 2A, 2B, 2C, and 2D depict a sequence whereby the embodiment is supplied with attractant and therapeutic agents in vivo, and captures and treats circulating cancer cells.

FIG. 2A depicts an embodiment of the device (1) comprised of a magnetic ring deployed within a patient's blood vessel (2). The device is being replenished via intravenous injection (5) with an attractant to attract circulating cancer cells (6). This attractant (6) consists of an agonist drug (to mimic chemokine proteins), bound with ferrous particles, and within a protective polymer that extends the attractant drug release over a prolonged period of time.

FIG. 2B depicts the attractant circulating cells (6) bound to the device (1) by magnetic force. Circulating cancer cells (4) are migrating down the blood vessel, attracted to the substance (6). The substance (6) may also attract circulating white blood cells (not depicted).

FIG. 2C depicts the circulating cancer cells (4) bound to the attractant substance (6) and captured by the device (1). A therapeutic agent (7) is being injected intravenously (5). The therapeutic agent (7) consists of ferrous microparticles coated with a minute quantity of a chemotherapy drug.

The attractant and/or therapeutic agent ferrous particle may also be magnetized, at the opposite polarity as the device, to enhance binding

FIG. 2D depicts the therapeutic agent (7) bound to the device (1) by magnetic force. The therapeutic agent (7) elutes the chemotherapy drug, destroying or degrading the captured cancer cells (4). White blood cells (not depicted) may also exert a therapeutic effect on the captured cancer cells (4). The destroyed cancer cells (4), therapeutic agent (7), and attractant substance (6) break down over time, leaving only ferrous microparticles attached to the device (1).

### EXAMPLE 3

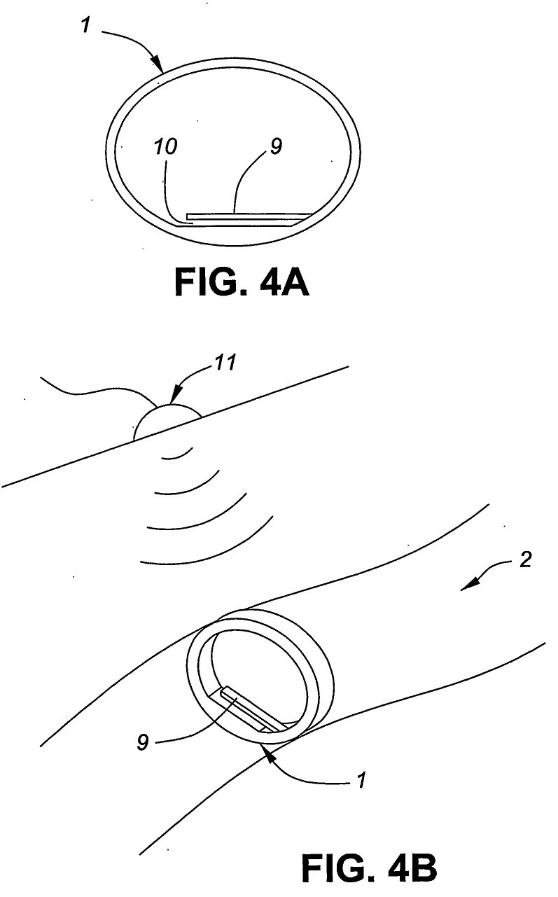

The device may alternatively incorporate a mechanical means to destroy or otherwise degrade captured cells. This would avoid the need to introduce therapeutic substances into the patient and would enable the destruction of captured cells to be done repeatedly as required. As depicted in FIG. 4A, the device (1) features a member (9) consisting of a cantilevered beam positioned over a portion of the surface of the device where the attractant is bound (10).

FIG. 4B depicts the device (1), with a mechanical means to destroy or degrade captured cells, deployed within a blood vessel (2). Once diseased cells have been attracted to and captured by the device, the member (9) can be set in motion under an external energy source (11) to impact upon and destroy or degrade the captured cells. The external energy source may be ultrasonic, magnetic or other transcutaneous energy delivery means. The cantilevered beam member (9) may have a specific geometry such that its harmonic frequency corresponds to a set frequency of a particular ultrasound and the ultrasound energy sets it in resonant vibration.

The foregoing has constituted a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention that have been described. They are not to be restricted to such variants, but are to be read as covering the full scope of the invention as is implicit within the invention and the disclosure that has been provided herein.

1-55. (canceled)

56. An implantable device for attracting circulating cells or foreign substances comprising:
a frame configured for attachment to a localized position in a body vessel, and
at least one attractant on the frame,
wherein the attractant is capable of attracting a diseased cell or a foreign substance.

57. An implantable device of claim 56, wherein the frame comprises a member for receiving the attractant.

58. An implantable device of claim 57, wherein the member forms a physical barrier between a cell lining a fluid vessel and the attractant.

59. An implantable device according claim 56, wherein the frame comprises magnetic components.

60. An implantable device according to claim 56, further including one or more of:
an endothelial cell;
an agonist equivalent of an endothelial cell,
a host cell,
an organ cell,
osseous tissue
a biotherapeutic, or
a chemical,
wherein an environment suitable for cell or foreign substance proliferation is created.

61. An implantable device according to claim 56, wherein the device or a portion of the device is shaped and/or coated to mimic the physiology of body organs.

62. An implantable device according to claim 56, wherein the device or a portion of the device has been modified to enhance its visibility to imaging systems including ultrasound, MRI, X-ray and CT systems.

63. An implantable device according to claim 56, wherein the attractant comprises one or more of:
a chemokine protein,
a viral host cell receptor proteins,
an agonist chemical for stimulating physiological activity at a cell receptor,
a chemokine agonist,
a bacterial toxin,
a foreign chemical substance, and
a combination of any one of (a) to (f)
and, optionally, wherein the attractant is in combination with a protective substance for protecting the attractant and for extending release.

64. An implantable device according to claim 63, wherein the protective substance is a polymer.

65. An implantable device according to claim 63, wherein the attractant includes a magnetic component, including a ferrous particle.

66. An implantable device according to claim 56, further including a container for containing and dispensing the attractant, and optionally, wherein said containment means dispenses attractant upon activation by an external energy source, said energy source including ultrasonic energy.

67. An implantable device according to claim 66, further including an attractant replenisher.

68. An implantable device according to claim 56 further including a therapeutic agent.

69. An implantable device according to claim 68, wherein the therapeutic agent comprises a chemical, a medicament, a chemotherapeutic medicament, an ionizing radiation source, and/or a mechanical agent.

70. An implantable device according to claim 69 wherein the mechanical agent comprises a cantilevered beam activatable vibrationally upon application of ultrasound at a resonant frequency.

71. An implantable device according to claim 69 wherein the mechanical agent includes an external energy source, including ultrasound, magnetism, or electricity.

72. An implantable device according to claim 68 wherein the therapeutic agent comprises an immune response stimulator, preferably, including an effective amount of an antigen, an interferon, an chemokine, a lymphokine, or a foreign substance.

73. An implantable device according to claim 68, wherein the therapeutic agent includes a magnetic component, including a ferrous particle.

74. An implantable device according to claim 68, further including a container for containing and dispensing the therapeutic agent, and optionally, wherein the container dispenses therapeutic agent upon activation by an external energy source such as ultrasonic energy.

75. An implantable device according to claim 68, further including a therapeutic agent replenisher.

76. A method of using the implantable device according to claim 56 as a cellular attractant, a viral attractant, and/or in the treatment of cancer.

77. A method of using of an implantable device, comprising:
removing a quantity of white blood cells from a body;
providing systemic chemotherapy treatment to the body;
deploying a device according to claim 72, within the body for capturing circulating diseased cells;
reintroducing the removed quantity of white blood cells into the body proximate to the device for use in degrading the captured circulating diseased cells.

78. An implantable device for attracting circulating cells or foreign substances comprising:
a frame configured for positioning in a localized position in a body vessel,
at least one attractant on the frame, and
at least one therapeutic agent on the frame,
wherein the attractant is capable of attracting a diseased cell or virus and wherein the therapeutic agent is capable of degrading the diseased cell or virus.

79. A method of attracting circulating cells and foreign substances, comprising:
deploying in a body vessel an implantable device for attracting circulating cells and foreign substances, the device comprising: a frame configured for attachment to a localized position in the body vessel, and
providing at least one attractant proximate to the frame, wherein the attractant is capable of attracting a diseased cell or foreign substance, said attractant optionally bound to the frame.

80. A method of claim 79, further comprising providing at least one therapeutic agent proximate to the frame, wherein the therapeutic agent is capable of degrading a diseased cell or foreign substance and, optionally, wherein the therapeutic agent is bound to the frame.

81. A method of claim 79, further comprising applying at least one therapeutic agent proximate to the frame, wherein the therapeutic agent is capable of degrading a diseased cell or foreign substance.

82. A method of claim 81, wherein the therapeutic agent is applied by local injection.

83. A method of claim 80, wherein the therapeutic agent is an ionizing radiation source and is directed to a location proximate to the frame.

84. An implantable device according to claim 80 wherein the therapeutic agent includes an external energy source and is directed to a location proximate to the frame, and, optionally, wherein said external energy source is ultrasound, a magnetic source or an electrical source.
